(19) United States
(12) Patent Application Publication
(10) Pub. No.: US 2022/0040655 A1
(43) Pub. Date: Feb. 10, 2022

(54) FLUORINATED AMINE OXIDE SURFACTANTS

(71) Applicant: 3M INNOVATIVE PROPERTIES COMPANY, St. Paul, MN (US)

(72) Inventors: Patricia M. Savu, Maplewood, MN (US); Nicholas L. Untiedt, Minneapolis, MN (US); Jason M. Kehren, Stillwater, MN (US)

(21) Appl. No.: 17/297,907

(22) PCT Filed: Dec. 12, 2019
(86) PCT No.: PCT/IB2019/060716

§ 371 Date: May 27, 2021

Related U.S. Application Data
(60) Provisional application No. 62/778,565, filed on Dec. 12, 2018.

(51) Int. Cl. B01F 17/00; C09G 1/02; C09K 13/08; C11D 1/00; C07C 311/09; C07D 295/26; C07D 295/13

(52) U.S. Cl. CPC B01F 17/0057; C09G 1/02; C09K 13/08; H01L 21/02068; C07C 311/09; C07D 295/26; C07D 295/13; C11D 1/004

(57) ABSTRACT

Compositions including one or more fluorochemical surfactants of the formula: (I) where R_{f} is a perfluoroalkyl group, each of R¹, R² and R³ are C₁-C₂₀ alkyl, alkoxy, or aryl; and R⁴ is alkylene, arylene of a combination thereof. R⁴ is preferably an alkylene of 1-20 carbons that may be cyclic or acyclic, may optionally contain catenated or terminal heteroatoms selected from the group consisting of N, O, and S. Most preferably R⁴ is an alkylene of 2-10 carbon atoms. Described are anionic N-substituted fluorinated amine oxide surfactants, and use thereof in cleaning and in acid etch solutions. The cleaning and etch solutions are used with a wide variety of substrates, for example, in the cleaning and etching of silicon oxide-containing substrates.

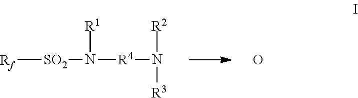

### FIELD OF THE INVENTION

The present invention is directed to certain fluorinated amine oxide surfactants, and use thereof in cleaning solutions, such as in aqueous etch solutions. The etch and cleaning solutions can be used with a wide variety of substrates in semiconductor processing like rinsing of silicon wafer during photolithography or in RCA cleaning solutions.

### BACKGROUND

The use of microelectronic devices, such as integrated circuits, flat panel displays and microelectromechanical systems, has burgeoned in new business and consumer electronic equipment, such as computers, laptops, e-readers, cell phones, and medical electronics. Such devices have also become an integral part of more established consumer products such as televisions, home appliances, and automobiles.

These devices in turn contain one or more very high-quality semiconductor chips containing many layers of circuit patterns. Many processing steps are required to convert a bare silicon wafer surface to a semiconductor chip of sufficient complexity and quality to be used, for example, in high performance logic devices found in personal computers.

The most common processing steps of semiconductor chip manufacture are wafer-cleaning steps, accounting for over 10% of the total processing steps. These cleaning steps are normally one of three types: oxidative, etch or penetrative flushing (or a combination of the three). During oxidative cleaning steps, oxidative compositions are used to oxidize the silicon or polysilicon surface, typically by contacting the wafer with aqueous peroxide or ozone solution. During etch cleaning steps, etching compositions are used to remove native and deposited silicon oxide films and organic contaminants from the silicon or polysilicon surface before gate oxidation or epitaxial deposition, typically by contacting the wafer with aqueous acid. See, for example, L. A. Zazzera and J. F. Moulder, J. Electrochem. Soc., 136, No. 2, 484 (1989). During penetrative flushing, water is used to remove all of the reagent that might be in the pattern features. In photolithographic processing water is used to remove all the tetramethylammonium hydroxide that might be in the pattern features in the step after the mask is developed with tetramethylammonium hydroxide in the previous step. The ultimate performance of the resulting semiconductor chip will depend greatly on how well each cleaning step has been conducted.

In the development of cleaning semiconductor wafers, several chemistries have been explored, and a few remain as the industry standards. These industry standards are known as Standard Clean-1 (SC-1; also known as RCA-1) and Standard Clean-2 (SC-2; also known as RCA-2). SC-1 has an alkaline pH and contains ammonium hydroxide (NH₄OH), hydrogen peroxide (H₂O₂) and water. Typically, SC-1 is used in the first step to remove metal ions and oxide surface organic materials. This procedure is then followed by application of SC-2, to remove heavy metals, alkalis and metal hydroxide contaminants. SC-2 has an acidic pH and contains hydrochloric acid (HCl), hydrogen peroxide and water. If a semiconductor wafer is heavily contaminated with organic material solutions of sulfuric acid (H₂SO₄) and hydrogen peroxide (H₂O₂) may be used. These solutions are called Piranha (see Burkman et al., Handbook of Semiconductor Wafer Cleaning Technology, Chapter 3, Aqueous Cleaning Processes; 120-3). Other materials that have been used to clean wafer surfaces include, but are not limited to, aqueous solutions of hydrogen fluoride (HF), hydrogen bromide (HBr), phosphoric acid, nitric acid, acetic acid, citric acid, ozone, and mixtures thereof.

### SUMMARY

The present invention provides a composition which includes one or more fluorochemical surfactants of the formula:

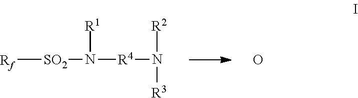

wherein R_{f} is a perfluoroalkyl group, each of R¹, R² and R³ are C₁-C₂₀ alkyl, alkoxy, or aryl; and R⁴ is alkylene, arylene of a combination thereof. R⁴ is preferably an alkylene of 1-20 carbons that that may be cyclic or acyclic, may optionally contain catenated or terminal heteroatoms selected from the group consisting of N, O, and S. Most preferably R⁴ is an alkylene of 2-10 carbon atoms.

Aqueous compositions are useful in cleaning substrates including cleaning or polishing silicon or GaAs, silicon or GaAs wafers coated with thin films of various compositions including metals, conductive polymers, insulating materials, and also copper-containing substrates, such as for example, copper interconnects.

In one aspect the present invention includes a composition including: (a) at least 10 ppm, typically from about 10 to about 10000 ppm of at least one surfactant of Formula I. The composition preferably employs water as a solvent. The composition may further include acid such as hydrochloric acid to make the media acidic or an alkaline material, for example, ammonium hydroxide, to make the medium basic.

A second aspect includes a method of cleaning a substrate comprising the steps of: (a) providing a composition as defined above; (b) providing a substrate comprising at least one surface, typically having at least one metal interconnect and/or film, the metal interconnect and/or film having at least one unwanted material on the surface; (c) bringing the surface of the substrate and the composition into contact with each other to form an interface; and (d) allowing removal of unwanted surface material.

Another aspect is an aqueous acid cleaning solution containing an acid; and a surfactant of Formula I. Typical acids include, but are not limited to, hydrogen fluoride, hydrogen chloride, nitric acid, sulfuric acid, phosphoric acid, acetic acid and/or citric acid. The solution optionally includes peroxide (e.g. hydrogen peroxide) or other additives such as ozone.

Still another embodiment of the present invention is an aqueous cleaning solution containing at least 0.001 wt. % of a surfactant of Formula I wherein the solution has a pH of 7 or greater.

Another aspect is an aqueous basic cleaning solution containing a base; and a surfactant of Formula 1. Typical bases include, but are not limited to, ammonium hydroxide, tetramethyl ammonium hydroxide, and/or tetrabutyl ammonium hydroxide. The solution optionally includes peroxide (e.g. hydrogen peroxide).

Still another embodiment of the present invention is an aqueous cleaning solution containing at least 10 ppm of surfactant of Formula 1 wherein the solution has a pH of 7 or less.

Still another embodiment of the present invention is an aqueous cleaning solution containing at least 10 parts per million (ppm) of a surfactant of Formula I in low ion water to be used in penetrative flushing.

The fluorinated surfactant is sufficiently stable in the aqueous acid solution, and advantageously reduces the surface tension thereof so that nanoscale features may be effectively produced on a silicon substrate, such as an integrated circuit and is soluble in the aqueous acid etch solutions and is low in metals so as to not be a source of contamination. The solution of the instant invention provides one or more of the following advantages: the solution has essentially the same etch rate as conventional etch solutions; possesses low surface tension; is low to non-foaming, can be filtered to remove particulates that may contaminate a substrate; leaves low or no surface residues on rinsing; is stable to extended storage; and affords excellent substrate surface smoothness. Other substrates, including metals and oxides may also be etched and cleaned by appropriate selection of acid or mixtures of acids.

In one aspect, this invention relates to a cleaning solution useful in semiconductor and integrated circuit manufacture, the composition including a fluorinated surfactant, ammonium hydroxide and hydrogen peroxide (SC-1). Advantageously, the present invention provides an aqueous etch solution useful for removal of residues such metals or organics, that contains a relatively low concentration of surfactant, but effectively wets the substrate and has an efficient rate of etching.

In another aspect, this invention relates to a cleaning solution useful in semiconductor and integrated circuit manufacture, the composition including a fluorinated surfactant, hydrogen chloride and hydrogen peroxide (SC-2). Advantageously, the present invention provides an aqueous etch solution useful for removal of residues such as heavy metals or metal hydroxides, that contains a relatively low concentration of surfactant, but effectively wets the substrate and has an efficient rate of etching.

In another aspect, this invention relates to an oxidative cleaning process for substrates by contacting a substrate with a homogeneous etch solution including the fluorinated surfactant, sulfuric acid and hydrogen peroxide (piranha solution). In a preferred embodiment, this invention relates to a cleaning process for substrates by contacting a substrate with a homogeneous etch solution including the fluorinated surfactant, sulfuric acid, and hydrogen peroxide for a time sufficient to achieve a predetermined degree of cleaning. The present invention provides a cleaning solution with low surface tension that easily penetrates the intricate microstructures and wets the surfaces on silicon substrates to destroy all organic residues.

In another aspect, this invention relates to an etching process for substrates by contacting a substrate with a homogeneous etch solution including the fluorinated surfactant and hydrogen fluoride to remove oxide layers and metal impurities. Optionally, the solution may include ammonium fluoride.

In another aspect, this invention relates to cleaning process for substrates by contacting the substrate with a homogeneous solution including the fluorinated surfactant in neutral low metal water (18 megaohm) for a time sufficient to achieve a predetermined degree of cleaning by flushing reagents and metals out of the depressed areas on the substrate.

The aqueous cleaning compositions may be prepared with very low metal content. Lower metal ion concentrations are preferred to minimize wafer surface contamination by the metal ions. As reported by Takahashi et al., metallic impurities account for over 50% of the yield losses in integrated circuit manufacture. See Determination of Trace metal Impurities in Semiconductor Grade Phosphoric Acid by high Sensitivity Reaction Cell ICP-MS, www.agilent.com/cs/library/applications/5988-8901EN.pdf

The present invention provides aqueous compositions comprising the instant surfactants and having an ionic species content, including metals, of less than 1,000 ppb, preferably less than 500 ppb, most preferably less than 200 ppb as measured by inductively coupled plasma (ICP). Distillation is an effective means of removing metal contaminants from these materials.

The term “alkyl” refers to straight or branched, cyclic or acyclic hydrocarbon radicals, such as methyl, ethyl, propyl, butyl, octyl, isopropyl, tert-butyl, sec-pentyl, and the like. Alkyl groups include, for example, 1 to 12 carbon atoms, 1 to 8 carbon atoms, or preferably 1 to 6 carbon atoms.

The term “perfluoroalkyl” refers to a fully fluorinated monovalent straight or branched, cyclic or acyclic, saturated hydrocarbon radical such as, for example, CF₃CF₂—, CF₃CF₂CF₂—, CF₃CF₂CF₂CF₂—, (CF₃)₂CFCF₂CF₂—, CF₃CF(CF₂CF₃)CF₂CF₂—, and the like. Perfluoroalkyl groups include, for example, 2 to 10 carbon atoms, preferably 3 to 5 carbon atoms, and most preferably 4 carbon atoms.

### DETAILED DESCRIPTION

With respect to the surfactant of Formula I, R_{f} is a C₂ to C₈ perfluoroalkyl group, preferably C₃-C₅; each of R¹, R² and R³ are C₁-C₈ alkyl, alkoxy or aryl. The alkyl groups are optionally interrupted by a catenary nitrogen atom. Preferably R¹ is an C₁-C₄ group. Preferably alkylene R² and R³ are independently C₁-C₄ groups. The alkylene defined by (CH₂)ₙ is optionally interrupted by a catenary oxygen atom; i.e. —C₄H₈—O—C₃H₆—.

It is expected that the surfactants of the present invention, which contain relatively short perfluoro alkali segments (less than 8 perfluorinated carbon atoms) when exposed to biological, thermal, oxidative, hydrolytic, and photolytic conditions found in the environment, will break down to functional, short chain fluorocarbon degradation products that will not bio-accumulate. For example, compositions of the present invention containing a perfluorobutyl moiety, such as CF₃CF₂CF₂CF₂— are expected to eliminate from the body much more effectively than perfluorooctyl. For this reason preferred embodiments of the R_{f} group in the above formula include perfluoroalkyl groups CₘF₂ₘ₊₁— containing a total of 3 to 5 carbon atoms.

The present invention provides aqueous compositions used for cleaning substrates and also used as an etch solutions. In some embodiments the compositions for cleaning substrates include at least one fluorinated surfactant of Formula I, an aqueous solvent and an oxidizing agent. The etch composition or solution is an aqueous solution containing an acid and at least one fluorinated surfactant.

The surfactant of Formula I may be prepared by reaction of a fluorinated sulfonyl halide with a diamine of the formula HNR¹—R⁴—NR²R³ to produce the aminoalkylsulfonamide compound:

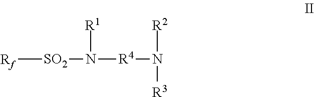

wherein R_{f} and R¹-R⁴ are as previously defined, followed by oxidation to the N-oxide. Alternatively, the intermediate II may be prepared by alkylation of a sulfonamide of Formula I where R¹=H to provide the requisite R¹ group. Useful oxidizing agents include hydrogen peroxide, percarboxylic acids, alkyl hydroperoxides, and ozone. Hydrogen peroxide is a preferred oxidizing agent (see, Kirk Othmer, 3rd ed., v. 2, pp. 259 to 271). Intermediate II can be distilled to reduce ionic contaminant (including metals) to the ppb level, the desired concentration for semiconductor manufacturing, and then converted in low ion vessels and solvents to the desired amine oxide that is an effective surfactant. Other related sulfonamide compounds of Formula I where R¹=H are not distillable and cannot achieve the low metal content of the instant surfactants.

Substrates useful in the present invention include silicon, germanium, GaAs, InP and other III-V and II-VI compound semiconductors. It will be understood, due to the large number of processing steps involved in integrated circuit manufacture, that the substrate may include layers of silicon, polysilicon, metals and oxides thereof, resists, masks and dielectrics. The present invention is also particularly useful in the etch and release of silicon-based microelectromechanical (MEMS) devices. The cleaning and drying of MEMS has similar issues to those for semiconductor chip manufacture. When the substrate is a copper interconnect, it is defined herein as a surface pattern containing copper. A film is defined herein as a thin coating of material on the substrate such as a silicon wafer, for example, a film of copper metal, silicon nitride, photoresist or a dielectric.

The compositions are aqueous and may include a water soluble organic solvent, specifically a polar organic solvent. A polar solvent is defined herein as having a dielectric constant greater than 5 at room temperature. Examples of suitable polar organic solvents include, but are not limited to, esters such as methyl formate, ethyl formate, methyl acetate, dimethyl carbonate, diethyl carbonate, propylene carbonate, ethylene carbonate, and butyrolactones (e.g., gamma butyrolactone); nitriles such as acetonitrile and benzonitrile; nitro compounds such as nitromethane or nitrobenzene; amides such as N,N-dimethylformamide, N,N-diethylformamide, and N-methylpyrrolidinone; sulfoxides such as dimethyl sulfoxide; sulfones such as dimethylsulfone, tetramethylene sulfone, and other sulfolanes; oxazolidinones such as N-methyl-2-oxazolidinone; alcohols such as ethanol and isopropanol and mixtures thereof.

A particularly suitable solvent is water, and in particular de-ionized water. A preferred polar organic solvent is isopropanol.

The compositions of the present invention are particularly useful for cleaning a substrate, e.g., silicon wafers and/or cleaning metal interconnects and/or film. Examples of polishing include, but are not limited to, chemical mechanical polishing (CMP), chemical enhanced polishing (CEP), and electrochemical mechanical deposition (ECMD). Examples of cleaning include, but are not limited to, wafer cleaning.

The present invention provides a method of cleaning a substrate comprising the steps of: (a) providing an aqueous composition containing: (i) at least 10 ppm of the surfactant of Formula I, ii) optionally a polar organic solvent; and (iii) a cleaning agent; (b) providing a substrate (c) contacting the substrate and the composition to facilitate removal of unwanted surface material.

The cleaning agent may include hydrofluoric acid, hydrochloric acid, nitric acid, sulfuric acid, phosphoric acid, acetic acid and/or citric acid, and the like, and is used in amounts of 0.1 to 98% in aqueous solution. Alternatively, the cleaning agent may include ammonium hydroxide, tetramethyl ammonium hydroxide and/or tetrabutyl ammonium hydroxide, and the like, and is used in amounts of 0.1 to 50% in aqueous solution. Optionally, the cleaning agent may include peroxide (e.g. hydrogen peroxide), ozone and/or other additives. In some preferred embodiments the composition comprises at least ten ppm of the surfactant of Formula I.

The unwanted materials to be removed include, but are not limited to, residues, films, and contaminants including organics, metals, metal hydroxides and metal oxides. Suitable substrates of the present invention include, but are not limited to, a silicon or GaAs wafer coated with thin films of various compositions including metals, conductive polymers, and insulating materials.

Other substrates, such as metals may also be cleaned by appropriate selection of the acid, base, or peroxide. The fluorinated surfactant effectively reduces the surface tension of the solution, allowing effective wetting of the substrate.

The compositions and methods of this invention can offer enhanced wetting, which is especially important in small geometry patterns and for features with large aspect ratios, reduced particulate contamination, and reduced surface roughness all of which may lead to improvements in manufacturing efficiency by lowering defects to increase wafer yield, by decreasing cleaning times to increase wafer production or by allowing for longer etch bath life by reducing filtration losses of surfactant.

The improved performance is due in part to the low surface tension of the cleaning solution due to the fluorinated surfactants used, which contributes to the improved wetting of the surfaces. The surface tensions of the cleaning solutions are generally less than 50 dynes/cm, preferably less than 30 dynes/cm and most preferably less than 25 dynes/cm when measured at 25° C.

The cleaning solution may be prepared by combining, in any order, the aqueous acid, base, or peroxide and the fluorinated surfactant. For oxidized silicon substrates, concentration of acid, base, or peroxide may vary widely, i.e. from 0.1 to 98 wt. %, depending on the substrate and the etch rate desired. Generally, the concentration of hydrofluoric acid, hydrochloric acid, nitric acid, sulfuric acid, phosphoric acid, acetic acid, citric acid, ammonium hydroxide, tetramethyl ammonium hydroxide, tetrabutyl ammonium hydroxide and/or hydrogen peroxide is from about 0.1 to 10 wt. %.

The invention provides a process for cleaning a substrate by contacting the substrate with the cleaning solution of the invention for a time and at a temperature sufficient to produce the desired degree of cleaning. Preferably, the substrate is an oxidized silicon substrate. Normally an oxidized silicon substrate is etched at 15 to 100° C. If desired, the cleaning process may further comprise the step of rinsing the cleaning solution from the etched substrate. In one embodiment, the solution may be rinsed with water, and preferably with deionized water. In another embodiment, the etch solution is slowly replaced with deionized water in a gradient cleaning process.

If desired, the solution may further include a second surfactant, in addition to the above described surfactant of the invention. Such second surfactants include both fluorinated and non-fluorinated surfactants such as are known in the etching art. Reference may be made to Kikuyama et al., IEEE Transactions on Semiconductor Manufacturing, Vol. 3, 1990, pp 99-108. Generally, the second surfactant may comprise 0 to 80 weight % of the total surfactant; the total amount of first and second surfactants comprising 10 to 10,000 parts per million.

In another embodiment, the disclosure provides and etch composition comprising 10-15% H₂O₂; 25-50% range H₂SO₄ and at least one 0.001 wt. % of the surfactant of Formula I.

The surfactant is used in amounts sufficient to reduce the surface tension of the solution to the desired degree. For wet etching of silicon substrates, the surfactant is generally used in amounts sufficient to reduce the surface tension of the resulting solution to 50 dynes/cm or less, preferably less than 30 dynes/cm and most preferably less than 25 dynes/cm when measured at 25° C. Generally, the solution contains 10 to 10,000 parts per million of surfactant, and is preferably 100 to 1000 parts per million. Below 10 parts per million the solution may not exhibit the desirable reduced surface tension and low contact angle on silicon substrate.

Other substrates may also be etched by appropriate selection of the acid or acid mixture. Gold, indium, molybdenum, platinum, and nichrome substrates may be etched with a mixture of hydrochloric and nitric acids. Aluminum substrates may be etched with a mixture of phosphoric and nitric acids, and may optionally include acetic acid as a buffer. Silicon substrates may be etched with a mixture of hydrofluoric, nitric, and acetic acids. In general, the fluorinated surfactant is used in amounts described for the cleaning, etching or flushing previously described. A SIRTL etch solution may be prepared using a mixture of chromium trioxide and hydrofluoric acid to determine defects in single crystal silicon.

Additional optional additives may include, for example, abrasive particles, acids (e.g., H₂SO₄, dilute aqueous HF, HCl), corrosion inhibitors (e.g., benzotriazoles, tolyltriazole (TTA)), chelating agents (e.g., ammonium citrate, iminodiacetic acid (IDA), EDTA), electrolytes (e.g., ammonium hydrogen phosphate), other surfactants, brighteners, levelers, etc. Typically the oxidizing agents are additives present in a concentration ranging from 10 to 100,000 ppm.

The present disclosure further provides compositions that may be used in RCA cleaning operations. In one embodiment the disclosure provides an aqueous surfactant composition comprising 0.001-0.5 wt. % of the surfactant of Formula I, 1-10, preferably 3-5 wt. % of NH₄OH, 1-10, preferably 3-5 wt. % of H₂O₂ and deionized water. (SC-1 cleaning composition).

In another embodiment the disclosure provides and aqueous cleaning composition comprising 0.001-0.5 wt. % of the surfactant of Formula I, 0.25-10, preferably 0.5-5 wt. % of HF and deionized water.

In yet another embodiment the disclosure provides an aqueous surfactant composition comprising 0.001-0.5 wt. % of the surfactant of Formula I, 1-10, preferably 4-6 wt. % range of HCl, wt. %, 1-10, preferably 3-5 wt. % of H₂O₂ and deionized water. (SC-2).

The above RCA cleaning compositions may be used sequentially for removal of the organic contaminants (organic clean+particle clean) with aqueous ammonium hydroxide/hydrogen peroxide, removal of the thin oxide layer using hydrogen fluoride dissolved in water, removal of ionic contamination (ionic clean) using aqueous HCl with hydrogen peroxide, followed by a water rinse.

The aqueous surfactant compositions may also be used in a CMP slurry composition comprising wt. % range of the surfactant of Formula I, wt. % range of an organic acid, wt. % range of H₂O₂, an abrasive particle, and optionally a polar organic solvent or aqueous solvent. The organic acid may be selected from citric acid, oxalic acid, succinic acid, and an alkyl sulfonic acid. For polishing applications, typically the compositions of the present invention either comprise abrasive particles or are used in combination with a fixed abrasive. Suitable abrasive particles include, but are not limited to, alumina, silica, and/or cerium oxide. Generally abrasive particles are present in a concentration ranging from about 3 to about 10 wt. 00. Fixed abrasives typically are abrasive particles fixed in a polymer.

For electrochemical mechanical deposition (ECMD) applications, the compositions of the present invention further comprise a copper salt, which may be any copper salt that is soluble in the solvent (i.e., typically the concentration of the copper cation is at least 0.10 M in the solvent). Suitable copper salts include, but are not limited to, copper imides, copper methides, copper organo-sulfonates, copper sulfates, or mixtures thereof. Copper salts are typically present in a concentration ranging from about 0.10 M to about 1.5 M in the solvent.

### EXAMPLES

Unless otherwise noted, all parts, percentages, ratios, etc. in the Examples and the rest of the specification are by weight. Unless otherwise indicated, all other reagents were obtained, or are available from fine chemical vendors such as Sigma-Aldrich Company, St. Louis, Mo., or may be synthesized by known methods. Table 1 (below) lists materials used in the examples and their sources.

TABLE 1
Materials List

| DESIGNATION | DESCRIPTION |
|---|---|
| dimethylaminopropylamine | Obtained from Sigma-Aldrich Chemical Company, St. |
| | Louis, MO |
| perfluorobutanesulfonyl fluoride | Obtained from 3M Company, Maplewood, MN |
| hexane | Obtained from VWR International, LLC. Radnor, PA |
| N,N′,N′- trimethylamino1,3- | Obtained from Pfaltz & Bauer Waterbury, CT |
| propylamine | |
| N, N- diethyl-N- | Obtained from Tokyo Chemical Industry Co., Ltd. Tokyo, |
| methylethylenediamine | Japan |
| N, N-diethyl-ethylenediamine | Obtained from Tokyo Chemical Industry Co., Ltd. Tokyo, |
| | Japan |
| 1-methylpiperazine | Obtained from Sigma-Aldrich Chemical Company, St. |
| | Louis, MO |
| 1-ethylpiperazine | Obtained from Alfa Aesar. Ward Hill, MA |
| aminoethyl-morpholine | Obtained from Alfa Aesar. Ward Hill, MA |
| N(iPr)₂Et | Diisopropyl ethyl amine, Obtained from Avocado |
| | Research Chemicals Limited London, UK. |
| dipropylsulfate | |
| dimethylsulfate | Obtained from Sigma-Aldrich Chemical Company, St. |
| | Louis, MO |
| Methyl tosylate | Obtained from Tokyo Chemical Industry Co., Ltd. Tokyo, |
| | Japan |
| Ethyl tosylate | Obtained from Alfa Aesar. Ward Hill, MA |
| 3-(Diethylamino)propylamine | Obtained from Sigma-Aldrich Chemical Company, St. |
| | Louis, MO |
| THF | Tetrahydrofuran, obtained from EMD Millipore |
| | Corporation. Billerica, MA |
| DCM | Dichloromethane, Obtained from EMD Millipore |
| | Corporation. Billerica, MA |
| MeCN | Acetonitrile, Obtained from EMD Millipore Corporation. |
| | Billerica, MA |
| Acetone | Obtained from EMD Millipore Corporation. Billerica, MA |
| NEt₃ | Triethylamine, obtained from EMD Millipore Corporation. |
| | Billerica, MA |
| K₂CO₃ | Potassium carbonate, obtained from VWR International, |
| | LLC. Radnor, PA |
| NaHCO₃ | Sodium Bicarbonate, obtained from Avantor Performance |
| | Materials, Center Valley, PA |
| Na₂SO₄ | Sodium sulfate, Obtained from Alfa Aesar. Ward Hill, MA |
| Ethyl Acetate | Obtained from VWR International, LLC. Radnor, PA |
| Heptane | Obtained from VWR International, LLC. Radnor, PA |
| Hexane | Obtained from VWR International, LLC. Radnor, PA |
| 30% hydrogen peroxide | Obtained from Avantor Performance Materials, Center |
| | Valley, PA |
| Isopropanol | Obtained from VWR International, LLC. Radnor, PA |
| PhMe | Toluene, Obtained from VWR International, LLC. Radnor, |
| | PA |
| DMF | Dimethylformamide, obtained from EMD Millipore |
| | Corporation. Billerica, MA |
| triethylene glycol monomethyl ether | Obtained from Tokyo Chemical Industry Co., Ltd. Tokyo, |
| | Japan |
| TsCl | p-Toluenesulfonyl chloride, Obtained from Alfa Aesar, |
| | Ward Hill, MA |
| 2-bromoethanol | Obtained from Pfaltz & Bauer Waterbury, CT |
| [dimethyl- | Obtained from Alfa Aesar. Ward Hill, MA |
| (trimethylsilylamino)silyl]methane | |
| MeI | Methyl iodide, Obtained from Alfa Aesar. Ward Hill, MA |
| nPrI | n-propyl iodide, Obtained from Sigma-Aldrich Chemical |
| | Company, St. Louis, MO |
| nPrBr | n-propyl bromide, Obtained from Oakwood Chemical, |
| | Estill, SC. |
| nBuI | n-butyl iodide, Obtained from Sigma-Aldrich Chemical |
| | Company, St. Louis, MO |
| nOctI | n-octyl iodide, Obtained from Sigma-Aldrich Chemical |
| | Company, St. Louis, MO |
| oxathiane 2,2-dioxide | 1,4-butane sultone, Obtained from Sigma-Aldrich |
| | Chemical Company, St. Louis, MO |

### Test Methods

### FPC Test Method

ICP samples were weighed out and then digested with concentrated nitric acid at 105° C., then diluted with water, and metals were measured on a Perkin Elmer 8300.

### Surface Tension Test Method

Samples were prepared at desired concentrations 250 parts per millions (ppm) or 2000 ppm (wt/wt) in 20 grams (g) of the following solvents: water, 2.5 wt % tetramethyl ammonium hydroxide, 2.5 wt % hydrochloric acid, or 50% sulfuric acid. Surface tensions were measured on a Kruss K100C, Analytical Instrument No. 1222. Surface tension is calibrated±1 dyne/cm.

NMR (¹H and ¹⁹F) Test Method

Samples were dissolved in CDCl₃, unless noted otherwise, and a Bruker FT NMR instrument was used to acquire ¹H NMR (500 MHz) and ¹⁹F NMR (500 MHz) spectra.

### LC/MS Test Method

The samples were analyzed by HPLC-HRMS using Agilent 6230 LC/MS TOF.

### EXAMPLES

### Synthesis of N-(dimethylamino)propyl)-1,1,2,2,3,3,4,4,4-nonfluorobutane-1-sulfonamide (1)

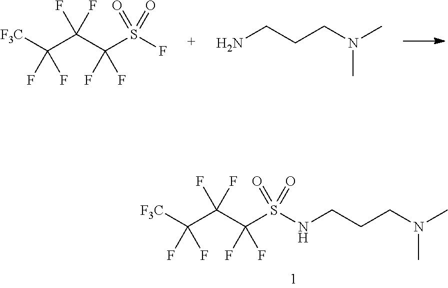

In a three-necked 2 L flask equipped with an overhead stirrer, thermocouple, addition funnel, and reflux condenser, dimethylaminopropylamine (642 g, 6.29 mol) and 2000 g of hexane was placed. With good agitation, fractionated perfluorobutanesulfonyl fluoride (992 g, 3.28 mol) was added over a period of an hour. The batch was then stirred at 50° C. for another 2 hours. After 2 hours a Dean-Stark trap (decanter assembly) was inserted between the flask and the condenser. The batch temperature was raised to 60° C., but no liquid was observed to condense in the trap. The flask was cooled to 47° C. and 500 mL of 18 megohm water was added to the reaction mixture.

The temperature of the flask contents was raised to 61° C. and 500 mL of hexane was stripped off. At this time another 500 mL of 18 megohm water was added. This was continued until 2000 mL of 18 megohm water had been added to the flask and the pot temperature had reached 70° C. and the hexane had been removed. At this time the flask contents became foamy and the flask was cooled to 21° C. The contents of the flask was allowed to settle for 15 minutes. At this time using a 4-inch-long 70 micron porous polyethylene rod (½ inch diameter), the liquid contents (water with dissolved dimethylaminopropylamine hydrofluoride) was siphoned out of the flask into another filter flask that had vacuum applied to it.

Another 2000 mL of 18 megohm water was added, the solid DMAP Amide was agitated with the water strongly for 30 minutes. The batch was allowed to settle and the water was removed as previously described.

Then another 2000 mL of 18 megohm water was added, the flask was agitated and the water was siphoned off to leave a wet cake of white solid in the flask. The white solid was shaken out and flushed with water into trays and the material was allowed to dry at room temperature overnight, followed by 3 hours at 100° C. A total of 1155 g of white solid was isolated. % Yield was 91% (1155/1261). The white powder was submitted for IPD and the results are reported in Table 2.

### Synthesis of N-Methyl-N-(dimethylamino)propyl)-1,1,2,2,3,3,4,4,4-nonfluoro-butane-1-sulfonamide (2)

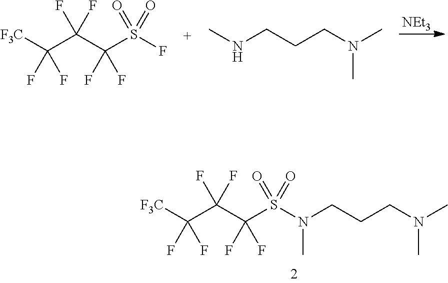

In a three-necked 1 L flask equipped with an overhead stirrer, thermocouple, addition funnel, and reflux condenser, fractionated perflurobutanesulfonyl fluoride (156 g, 0.516 mol) was placed. With good agitation, N,N′,N′-trimethylamino1,3-propylamine (60 g, 0.517 mol) and triethylamine (52 g, 0.514 mol) was added over a period of an hour. The batch was then stirred at 71° C. for another 2 hours. At the end of this time, the batch was cooled to room temperature, and 350 mL of water was added. The lower fluorochemical layer was split off and washed again with 600 mL of water to give 191 g of crude fluorochemical. The crude fluorochemical was distilled under vacuum (19.5 mm) to give 101 g of clear liquid material that distilled at a head temperature of 142-144° C. GC-MS was consistent for the desired material (2). The distilled material was submitted for ICP for level of metal ions.

Table 2 contains the results. As you can see in Table 2, compound 2 had total metals at 261 parts per billion (ppb) while the analogous material with a hydrogen on the sulfonamide nitrogen that was purified by washing with large amounts of 18 megohm water had metals had metals at 9790 ppb. Compound 1 was not easily distilled because it had a melting point of 140° C.; its analog with a methyl on the sulfonamide nitrogen had a melting point of −1° C. as determined by differential scanning calorimetry.

TABLE 2
Parts Per Billion Metals in Amides by ICP

| | ppb (precipitated 3X) (1) | ppb (distilled) (2) |
|---|---|---|
| Element | 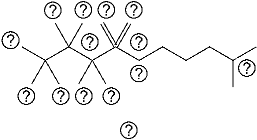 | 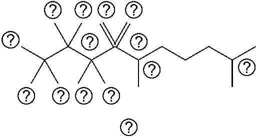 |
| Al | 400 | nd |
| As | nd | nd |
| Ba | 119 | nd |
| Ca | nd | 36 |
| Cd | nd | nd |
| Cr | 39 | nd |
| Cu | nd | nd |
| Fe | 1001 | nd |
| K | 4100 | nd |
| Li | 100 | nd |
| Mg | nd | nd |
| Mn | 11 | nd |
| Mo | nd | nd |
| Na | 3900 | 32 |
| Ni | 50 | nd |
| Pb | nd | nd |
| Sn | nd | nd |
| Sr | nd | nd |
| Ti | 60 | nd |
| V | nd | nd |
| Zn | nd | 193 |
| Zr | nd | nd |
| Total above | 9790 ppb | 261 ppb |

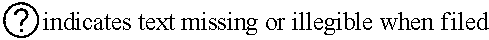

nd = not detected.

### PREPARATIVE EXAMPLES

### Synthesis of (N-Methyl-N-(diethylamino)ethyl)-1,1,2,2,3,3,4,4,4-nonfluorobutane-1-sulfonamide (3)

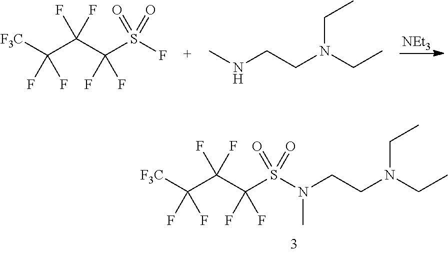

In a three-necked 1 L flask equipped with an overhead stirrer, thermocouple, addition funnel, and reflux condenser, N,N-diethyl-N-methylethylenediamine (81 g, 0.62 mol), triethyl amine (89 g, 0.80 mol) and 300 g of hexane was placed. With good agitation, fractionated perfluorobutanesulfonyl fluoride (188 g, 0.62 mol) was added over a period of an hour. The batch was then stirred at 50° C. for another 2 hours. After 2 hours a Dean-stark trap (decanter assembly) was inserted between the flask and the condenser. The flask was cooled to 47° C. and 500 mL of water was added to the reaction mixture. The temperature of the flask contents was raised to 61° C. and 500 ml of hexane was stripped off Another 500 mL of water was added, and the batch was split at 60° C. The lower fluorochemical phase was washed with another 1000 mL water. The lower fluorochemical phase was then distilled at vacuum (7.9-8.6 mm, 125-135° C.) to give 126 g of material that was a yellow solid. GC-MS of the distilled material confirmed that it was desired product (3) with a parent ion of 412. The melting point was determined by DSC and found to be 3° C. ¹H and ¹⁹F NMR data was consistent with the desired compound.

### Synthesis of N-(diethylamino)ethyl)-1,1,2,2,3,3,4,4,4-nonfluoro-butane-1-sulfonamide (4)

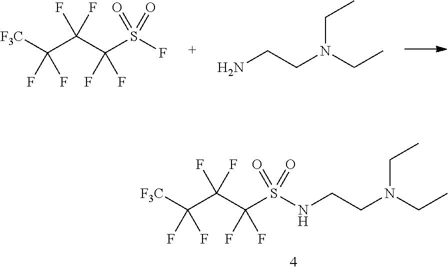

In a three-necked 1 L flask equipped with an overhead stirrer, thermocouple, addition funnel, and reflux condenser, N, N-diethyl-ethylenediamine (383 g, 3.31 mol), and 700 g of hexane was placed. With good agitation, fractionated perfluorobutanesulfonyl fluoride (500 g, 1.656 mol) was added over a period of an hour. The batch was then stirred at 50° C. for another 2 hours. After 2 hours a Dean-stark trap (decanter assembly) was inserted between the flask and the condenser. The flask was cooled to 47° C. and 1000 mL of water was added to the reaction mixture. The temperature of the flask contents was raised to 61° C. and 500 mL of hexane was stripped off. Another 500 mL of water was added, and the batch was split at 60° C. The lower fluorochemical phase was washed with another 1500 mL water. The lower fluorochemical phase was then distilled at vacuum (4.7-6.8 mm, 140-147° C.) to give 580 g of material. GC-MS of the distilled material confirmed that it was desired product (4) with a parent ion of 398. The melting point was determined by DSC and found to be 101° C.

### Synthesis of N-((4N′-Methyl)-piperazino)-1,1,2,2,3,3,4,4,4-nonfluorobutane-1-sulfonamide (5)

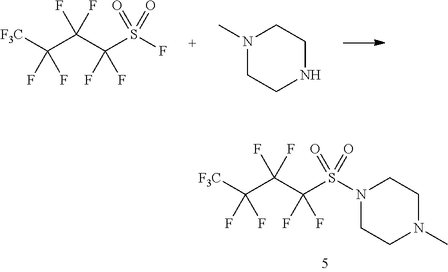

In a three-necked 1 L flask equipped with an overhead stirrer, thermocouple, addition funnel, and reflux condenser, 1-methylpiperazine (133 g, 1.32 mol), and 400 g of hexane was placed. With good agitation, fractionated perfluorobutanesulfonyl fluoride (200 g, 0.662 mol) was added over a period of an hour. The batch was then stirred at 50° C. for another 2 hours. After two hours a Dean-Stark trap (decanter assembly) was inserted between the flask and the condenser. The flask was cooled to 47° C. and 500 mL of water was added to the reaction mixture. The temperature of the flask contents was raised to 61° C. and 500 mL of hexane was stripped off. Another 500 mL of water was added and the batch was split at 60° C. The lower fluorochemical phase was washed with another 1000 mL water. The lower fluorochemical phase (212 g) was then distilled at vacuum (8.5 mm head temperature is 120° C.) to give 21 g of precut and then (6.7-7.9 mm, 110-125° C.) to give 178 g of heart cut material that was initially a liquid. GC-MS of the distilled material confirmed that it was desired product (5) with a parent ion of 382. The melting point was determined by DSC and found to be 23° C.

### Synthesis of N-((4N′-Ethyl)-piperazino)-1,1,2,2,3,3,4,4,4-nonfluorobutane-1-sulfonamide

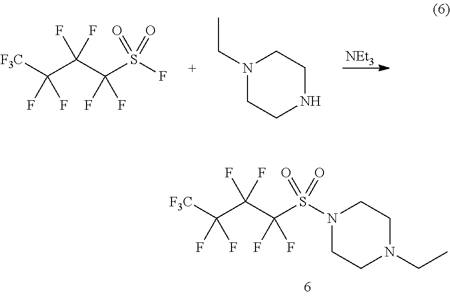

In a three-necked 2 L flask equipped with an overhead stirrer, thermocouple, addition funnel, and reflux condenser, 1-ethylpiperazine (195 g, 1.71 mol), and triethylamine (172 g, 1.71 mol) were placed. With good agitation, fractionated perfluorobutanesulfonyl fluoride (500 g, 1.66 mol) was added over a period of an hour. The batch was then stirred at 70° C. for another 2 hours. After 2 hours 900 mL of water was added. The lower fluorochemical phase (640 g) was then washed twice with another 1000 mL water to give 564 g of lower fluorochemical phase. The lower fluorochemical phase was distilled at vacuum 20-24 mm head temperature at 122-124° C. to give 17 g of precut and then (5.7-8.5 mm, 122-124° C.) to give 519 g of heart cut material that was a liquid. GC-MS of the distilled material confirmed that it was desired product (6) with a parent ion of 396. The melting point was determined by DSC and found to be 8° C.

### Synthesis of N-((4N′-Propyl)-piperazino)-1,1,2,2,3,3,4,4,4-nonfluorobutane-1-sulfonamide (8)

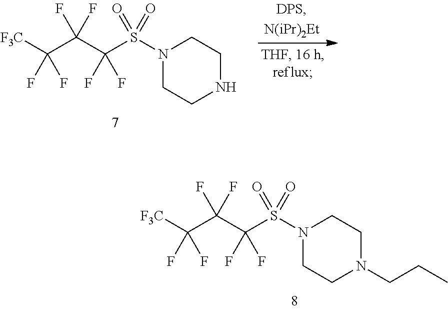 [SMILES: O=S(=O)(N1CCNCC1)C(F)(F)C(F)(F)C(F)(F)C(F)(F)F ; CCCN1CCN(S(=O)(=O)C(F)(F)C(F)(F)C(F)(F)C(F)(F)F)CC1]

1-((perfluorobutyl)sulfonyl)piperazine (7) was prepared as in WO2017/100045A: PE-1, page 15 lines 5-23.

A 250 mL round bottom flask with a condenser was charged with 1-((perfluorobutyl)sulfonyl)piperazine (7) (10.0 g, 27.2 mmol), THF (55 mL), and N(iPr)₂Et (4.0 g, 5.4 mL, 31 mmol). While stirring vigorously dipropylsulfate (5.0 g, 4.5 mL, 27 mmol) was added and the reaction was refluxed for 16 hours after which the reaction was cooled, diluted with 250 mL H₂O and diluted with 250 mL ethyl acetate, layers were separated and the organic was washed with brine, then dried over Na₂SO₄, filtered, and concentrated under vacuum to give a light yellow oil (8) (11.0 g, 26.8 mmol, 99% yield). The melting point was determined by DSC and found to be 29° C. ¹H and ¹⁹F NMR data was consistent with the desired compound.

### Synthesis of N-((aminoethyl)morpholino)-1,1,2,2,3,3,4,4,4-nonfluorobutane-1-sulfonamide (9)

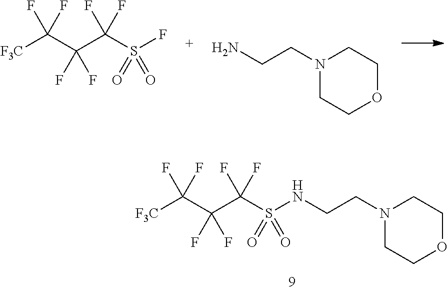 [SMILES: O=S(=O)(F)C(F)(F)C(F)(F)C(F)(F)C(F)(F)F ; NCCN1CCOCC1 ; O=S(=O)(NCCN1CCOCC1)C(F)(F)C(F)(F)C(F)(F)C(F)(F)F]

In a three-necked 2 L flask equipped with an overhead stirrer, thermocouple, addition funnel, and reflux condenser, aminoethyl-morpholine (342 g, 2.66 mol), and 600 g of hexane was placed. With good agitation, fractionated perfluorobutanesulfonyl fluoride (402 g, 1.33 mol) was added over a period of an hour. The batch was then stirred at 60° C. for another 2 hours. After 2 hours a Dean-Stark trap (decanter assembly) was inserted between the flask and the condenser. The hexane and unreacted PBSF was stripped to the Dean-Stark trap until the pot reached 92° C., then 500 mL of water was added keeping the batch temperature above 86° C. or otherwise the solid would start to form and impede washing. The washing was repeated with 500 mL of water two more times. The batch was stripped at atmospheric pressure until the pot reached 140° C. It was attempted to distill the material at reduced pressure, but vacuum was lost often by material subliming into the vacuum traps and hoses. The material was poured out into ajar hot and weighed (427 g). The melting point was determined by DSC and found to be 94° C.

### Synthesis of N-(diethylamino)propyl)-1,1,2,2,3,3,4,4,4-nonfluoro-butane-1-sulfonamide (10)

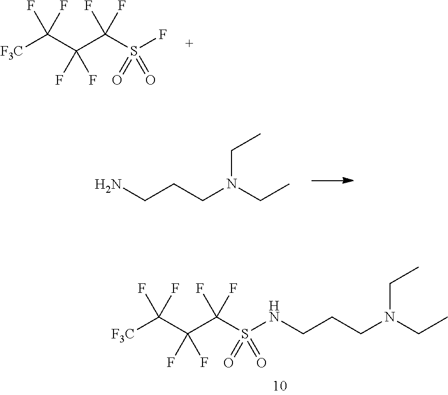

In a three-necked 2 L flask equipped with an overhead stirrer, thermocouple, addition funnel, and reflux condenser, 3-(Diethylamino)propylamine (480 g, 3.31 mol), and 700 g of hexane was placed. With good agitation, fractionated perfluorobutanesulfonyl fluoride (500 g, 1.656 mol) was added over a period of a hour. The batch was then stirred at 50° C. for another 2 hours. After 2 hours a Dean-Stark trap (decanter assembly) was inserted between the flask and the condenser. The flask was cooled to 47° C. and 1000 mL of water was added to the reaction mixture. The temperature of the flask contents was raised to 61° C. and 500 ml of hexane was stripped off Another 500 mL of water was added, and the batch was split at 60° C. The lower fluorochemical phase was washed with another 1500 mL water. The lower fluorochemical phase was then distilled at vacuum (5.7-7.0 mm, 148-151° C.) to give 605 g of yellow solid material. GC-MS of the distilled material confirmed that it was desired product (10) with a parent ion of 412. The melting point was determined by DSC and found to be 86° C.

### Synthesis of N-[3-(diethylamino)propyl]-1,1,2,2,3,3,4,4,4-nonafluoro-N-methyl-butane-1-sulfonamide (37)

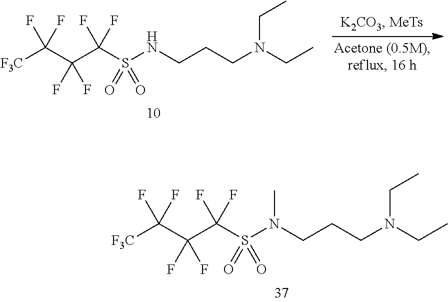 [SMILES: CCN(CC)CCCNS(=O)(=O)C(F)(F)C(F)(F)C(F)(F)C(F)(F)F ; CCN(CC)CCCN(C)S(=O)(=O)C(F)(F)C(F)(F)C(F)(F)C(F)(F)F]

A 250 mL round bottom flask was charged with N-[3-(diethylamino)propyl]-1,1,2,2,3,3,4,4,4-nonafluoro-butane-1-sulfonamide (10, 10.0 g, 24.3 mmol), K₂CO₃ (7.0 g, 51 mmol), and 120 mL acetone. While stirring, methyl tosylate (4.70 g, 25.2 mmol) was added, and the reaction was stirred at room temperature for 16 hours. The reaction was then diluted with 300 mL H₂O, the organic layer was separated, the aqueous layer was washed with 50 mL DCM twice, combined organics were dried over Na₂SO₄, filtered, concentrated under vacuum, and distilled under vacuum (95° C. @ 150 mTorr) to give a clear colorless liquid (37, 6.8 g, 66% Yield). ¹H and ¹⁹F NMR data was consistent with the desired compound.

### Synthesis of N-(3-(diethylamino)propyl)-N-ethyl-1,1,2,2,3,3,4,4,4-nonafluorobutane-1-sulfonamide (38)

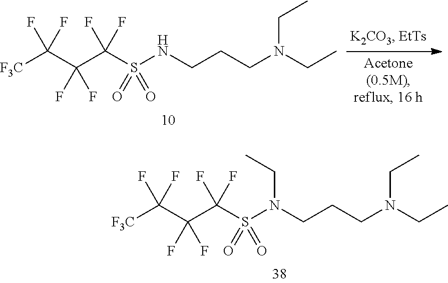 [SMILES: CCN(CC)CCCNS(=O)(=O)C(F)(F)C(F)(F)C(F)(F)C(F)(F)F ; CCN(CC)CCCN(CC)S(=O)(=O)C(F)(F)C(F)(F)C(F)(F)C(F)(F)F]

A 1 L three-necked flask equipped with a thermocouple, overhead stirrer, and an addition funnel was charged with N-[3-(diethylamino)propyl]-1,1,2,2,3,3,4,4,4-nonafluoro-butane-1-sulfonamide (10, 36.0 g, 97.1 mmol), K₂CO₃ (12.7 g, 101.9 mmol), and 150 mL acetone. While stirring, ethyl tosylate (18.4 g, 101.9 mmol) dissolved in 30 mL of acetone was added, and the reaction was stirred at reflux for 16 hours. The batch was cooled to room temperature and filtered with #4 filter paper using a Buchner funnel. The cake in the Buchner funnel was flushed with an additional 100 mL of acetone. The acetone was stripped off at atmospheric pressure until the pot temperature reached 89° C. to give 46 g. The remainder was distilled under vacuum (112° C. at 500 mTorr) to give a clear colorless liquid (38, 30.6 g, 80% Yield). GC/MS was consistent for the desired structure. ¹H and ¹⁹F NMR data was consistent with the desired compound.

### Synthesis of 2-(2-(2-methoxyethoxy)ethoxy)ethyl 4-methylbenzenesulfonate (39)

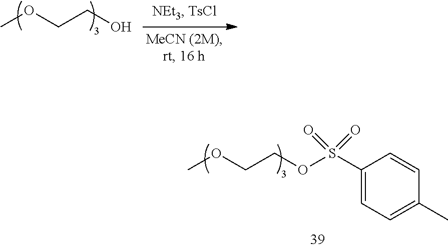

A 250 mL round bottom flask was charged with triethylene glycol monomethyl ether (15 g, 91 mmol), MeCN (46 mL), and NEt₃ (9.5 g, 13 mL, 95 mmol) and cooled to 5° C. in an ice/water bath. TsCl (17 g, 89 mmol) was added slowly over 10 min and the reaction was then removed from the ice bath and allowed to stir overnight at room temperature. The resulting mixture was then filtered, diluted with 100 mL ethyl acetate, washed twice with 50 mL sat. NaHCO₃ solution, washed with 100 mL H₂O, and 100 mL brine solution. The organic phase was dried over Na₂SO₄, concentrated to give a clear light-yellow oil (26.0 g, 81.7 mmol, 92% yield) that was used without further purification. ¹H NMR data was consistent with the desired compound.

### Synthesis of N-(3-(diethylamino)propyl)-1,1,2,2,3,3,4,4,4-nonafluoro-N-(2-(2-(2-methoxyethoxy)ethoxy)ethyl) butane-1-sulfonamide (40)

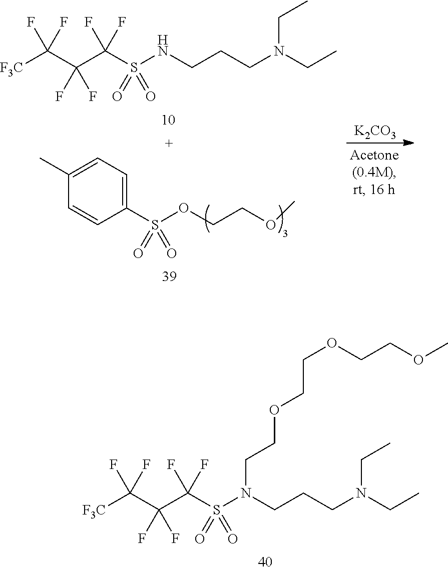

A 250 mL round bottom flask N-[3-(diethylamino)propyl]-1,1,2,2,3,3,4,4,4-nonafluoro-butane-1-sulfonamide (N-[3-(diethylamino)propyl]-1,1,2,2,3,3,4,4,4-nonafluoro-butane-1-sulfonamide (10, 10.0 g, 24.3 mmol), K₂CO₃ (7.0 g, 51 mmol), and 60 mL acetone. While stirring, the tosylate (39, 8.0 g, 26.6 mmol) was added and the reaction was stirred at room temperature for 16 hours. The reaction was then filtered and the filtrate was concentrated dissolved in heptane and filtered a second and the concentrated to give an orange slurry. The material was then distilled (160° C. @ 40 mTorr) to isolate the alkylated sulfonamide (40, 7.0 g, 52% yield) as clear colorless oil. ¹H and ¹⁹F NMR data was consistent with the desired compound.

### Synthesis of 2-bromoethoxy(trimethyl)silane (41)

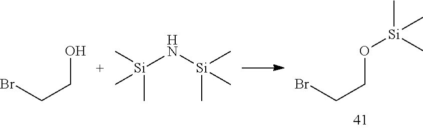

A 250 mL round bottom flask was cooled to 0° C. in and ice water bath and charged with 2-bromoethanol (20.0 g, 11.3 mL, 160 mmol) and [dimethyl-(trimethylsilylamino)silyl]methane (14.0 g, 18.1 mL, 160 mmol) the reaction was removed from the ice bath and stirred at room temperature, after 2 h the reaction was deemed complete by NMR and was filtered through a plug of celite to give crude 2-bromoethoxy(trimethyl)silane (41, 26.9 g, 85% yield) that was used without further purification. ¹HNMR data was consistent with the desired compound.

### Synthesis of N-(3-(diethylamino)propyl)-1,1,2,2,3,3,4,4,4-nonafluoro-N-(2-hydroxyethyl)butane-1-sulfonamide (42)

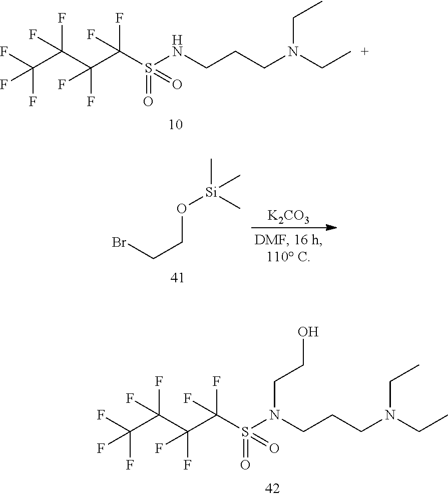

A 500 mL flask was charged with sulfonamide (10, 37.0 g, 89.7 mmol), 2-bromoethoxy(trimethyl)silane (41, 26.9 g, 136 mmol), K₂CO₃ (14.0 g, 101 mmol), and 180 mL DMF and heated to 110° C. overnight. The reaction was cooled to room temperature, quenched with 100 mL NH₄Cl solution and 100 mL H₂O. Then extracted with 200 mL ethyl acetate, washed with another 100 mL ethyl acetate, then washed the organics with 100 mL H₂O twice, the organic layer was dried over Na₂SO₄, filtered and the light brown oil was distilled (125-130° C. @ 20 mTorr) to obtain a clear colorless syrup (42, 28 g, 68%). ¹H and ¹⁹F NMR data was consistent with the desired compound.

### Synthesis of N-(3-(dimethylamino)-2,2-dimethylpropyl)-1,1,2,2,3,3,4,4,4-nonafluorobutane-1-sulfonamide (43)

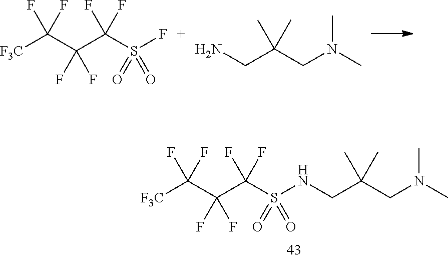

In a three-necked 1 L flask equipped with an overhead stirrer, thermocouple, addition funnel, and reflux condenser, tetramethylpropanediamine (172 g, 1.32 mol), and 352 g of hexane was placed. With good agitation, fractionated perfluorobutanesulfonyl fluoride (200 g, 0.622 mol) was added over a period of an hour. The batch was then stirred at 50° C. for another 2 hours. After two hours a Dean-stark trap (decanter assembly) was inserted between the flask and the condenser. The flask was cooled to 47° C. and 500 mL of water was added to the reaction mixture. The temperature of the flask contents was raised to 61° C. and 500 mL of hexane was stripped off. Another 500 mL of water was added and the batch was split at 60° C. The lower fluorochemical phase was washed with another 1000 mL water. The lower fluorochemical phase was then distilled at vacuum (16.5-19 mm, 132-140° C.) to give 185 g of material that was a yellow solid. GC-MS was consistent for the desired structure.

### Synthesis of N-(3-(dimethylamino)-2,2-dimethylpropyl)-1,1,2,2,3,3,4,4,4-nonafluoro-N-methylbutene-1-sulfonamide (44)

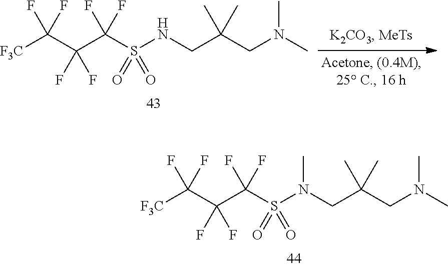 [SMILES: CN(C)CC(C)(C)CNS(=O)(=O)C(F)(F)C(F)(F)C(F)(F)C(F)(F)F ; CN(C)CC(C)(C)CN(C)S(=O)(=O)C(F)(F)C(F)(F)C(F)(F)C(F)(F)F]

A 250 mL round bottom flask was charged with sulfonamide (43, 10.0 g, 24.3 mmol), K₂CO₃ (7.0 g, 51 mmol), 120 mL acetone, and methyl tosylate (4.9 g, 26 mmol), this was stirred for 16 hours at room temperature after which 250 mL of water was added and the reaction was stirred for ˜20 min, then the resulting white solid was filtered, and dried under vacuum (44, 9.6 g, 93% Yield). ¹H and ¹⁹F NMR data was consistent with the desired compound.

### Synthesis of Materials in Tables 3, 4, and 5

### Synthesis of (N-Methyl-N-(diethylamino)ethyl)-1,1,2,2,3,3,4,4,4-nonfluorobutane-1-sulfonamido oxide (11)

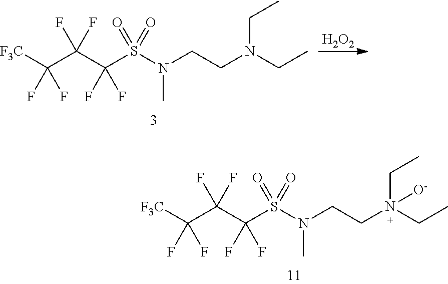

In a three-necked 1 L flask equipped with an overhead stirrer, thermocouple, addition funnel, and reflux condenser, of (N-Methyl-N-(diethylamino)ethyl)-1,1,2,2,3,3,4,4,4-nonfluorobutane-1-sulfonamide (3, 40 g, 0.97 mol), and 50 g of isopropanol was placed. The flask was heated to 67° C. With good agitation, 30% hydrogen peroxide (22 g, 0.21 mol) was added over a period of an hour. The flask was heated with good stirring overnight at 67° C. H-, F, and C-NMR showed the solution was 36% the desired (N-Methyl-N-(diethylamino)ethyl)-1,1,2,2,3,3,4,4,4-nonfluorobutane-1-sulfonamido oxide (11). LC/MS (negative electrospray) data and ¹H and ¹⁹F NMR data were consistent with the desired compound.

### Synthesis of N-((4N′-Methyl)-piperazino)-1,1,2,2,3,3,4,4,4-nonfluorobutane-1-sulfonamido amine oxide (12)

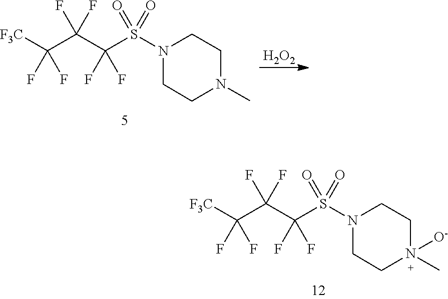

In a three-necked 1 L flask equipped with an overhead stirrer, thermocouple, addition funnel, and reflux condenser, of N-((4N′-Methyl)-piperazino)-1,1,2,2,3,3,4,4,4-nonfluorobutane-1-sulfonamide (5, 25 g, 0.0652 mol), and 50 g of isopropanol was placed. The flask was heated to 78° C. With good agitation, 30% hydrogen peroxide (15 g, 0.1305 mol) was added over a period of an hour. The flask was heated with good stirring overnight at 80° C. At the end of this time. The contents of the flask were poured out and dried in a vacuum oven to give 27 g of while solid. LC/MC negative ionization gave a mass of 457 (M+CH3CO2-) which gives a MW of 398.

### Synthesis of N-((4N′-Ethyl)-piperazino)-1,1,2,2,3,3,4,4,4-nonfluorobutane-1-sulfonamido Oxide (13)

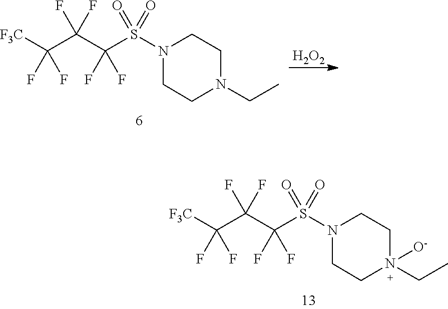

In a three-necked 1 L flask equipped with an overhead stirrer, thermocouple, addition funnel, and reflux condenser, N-((4N′-Ethyl)-piperazino)-1,1,2,2,3,3,4,4,4-nonfluorobutane-1-sulfonamide (6, 144 g, 0.363 mol), and 181.5 g of isopropanol was placed. The flask was heated to 80° C. With good agitation, 30% hydrogen peroxide (82.5 g, 0.727 mol) was added over a period of 2 hours. The flask was heated with good stirring overnight at 80° C. LC/MS (negative electrospray) data was consistent with the desired compound.

### Synthesis of N-((4N′-Propyl)-piperazino)-1,1,2,2,3,3,4,4,4-nonfluorobutane-1-sulfonamido Oxide (14)

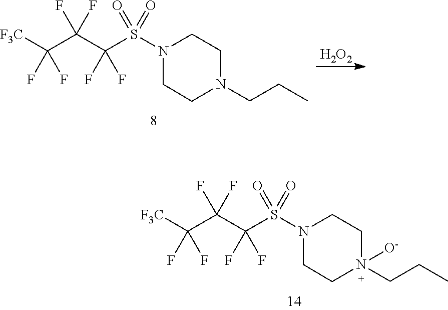

In a three-necked 1 L flask equipped with an overhead stirrer, thermocouple, addition funnel, and reflux condenser, N-((4N′-Propyl)-piperazino)-1,1,2,2,3,3,4,4,4-nonfluorobutane-1-sulfonamide (8, 10.5 g, 0.02625 mol), and 20 g of isopropanol was placed. The flask was heated to 80° C. With good agitation, 30% hydrogen peroxide (20.0 g, 0.176 mol) was added over a period of 2 hours. The flask was heated with good stirring overnight at 80° C. LC/MS (negative electrospray) data was consistent with the desired compound.

### Synthesis of Synthesis of N-Methyl-N-(dimethylamino)propyl)-1,1,2,2,3,3,4,4,4-nonfluoro-butane-1-sulfonamido oxide (15)

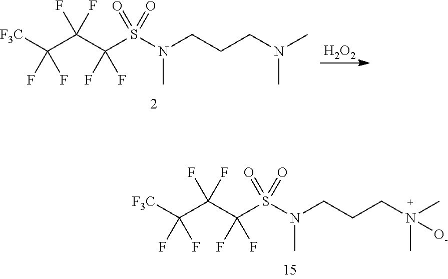

In a three-necked 1 L flask equipped with an overhead stirrer, thermocouple, addition funnel, and reflux condenser, N-Methyl-N-(dimethylamino)propyl)-1,1,2,2,3,3,4,4,4-nonfluoro-butane-1-sulfonamide (2, 40 g, 0.10 mol), and 50 g of isopropanol was placed. The flask was heated to 80° C. With good agitation, 30% hydrogen peroxide (22.7 g, 0.200 mol) was added over a period of 2 hours. The flask was heated with good stirring overnight at 80° C. LC/MS (negative electrospray) data was consistent with the desired compound.

### Synthesis of N-[3-(diethylamino)propyl]-1,1,2,2,3,3,4,4,4-nonafluoro-N-methyl-butane-1-sulfonamido oxide (16)

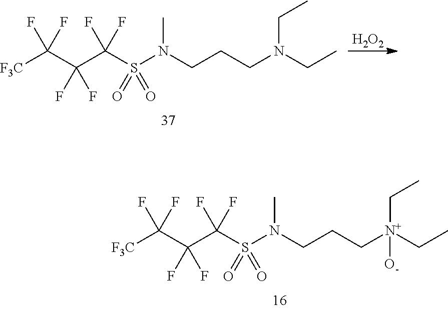

In a three-necked 1 L flask equipped with an overhead stirrer, thermocouple, addition funnel, and reflux condenser, N-(3-(diethylamino)propyl)-N-ethyl-1,1,2,2,3,3,4,4,4-nonafluorobutane-1-sulfonamide (37, 6.0 g, 45 mmol), and 20 g of isopropanol was placed. The flask was heated to 80° C. With good agitation, 30% hydrogen peroxide (4.0 g, 35 mmol) was added over a period of 45 minutes. The flask was heated with good stirring overnight at 80° C. LC/MS (negative electrospray) data was consistent with the desired compound.

### Synthesis of N-Ethyl-N-(diethylamino)propyl)-1,1,2,2,3,3,4,4,4-nonfluoro-butane-1-sulfonamido oxide (17)

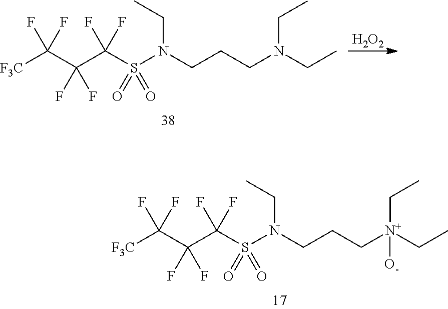

In a three-necked 1 L flask equipped with an overhead stirrer, thermocouple, addition funnel, and reflux condenser, N-(3-(diethylamino)propyl)-N-ethyl-1,1,2,2,3,3,4,4,4-nonafluorobutane-1-sulfonamide (38, 20.0 g, 0.0454 mol), and 30 g of isopropanol was placed. The flask was heated to 80° C. With good agitation, 30% hydrogen peroxide (11.0 g, 0.0969 mol) was added over a period of 2 hours. The flask was heated with good stirring overnight at 80° C. LC/MS (negative electrospray) data was consistent with the desired compound.

### Synthesis of N-(3-(diethylamino)propyl)-1,1,2,2,3,3,4,4,4-nonafluoro-N-(2-(2-(2-methoxyethoxy)ethoxy)ethyl) butane-1-sulfonamido oxide (18)

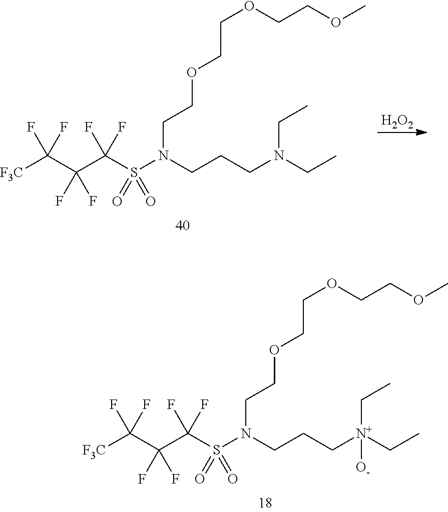

In a three-necked 1 L flask equipped with an overhead stirrer, thermocouple, addition funnel, and reflux condenser, N-(3-(diethylamino)propyl)-1,1,2,2,3,3,4,4,4-nonafluoro-N-(2-(2-(2-methoxyethoxy)ethoxy)ethyl) butane-1-sulfonamide (40, 6.0 g, 0.012 mol), and 14 g of isopropanol was placed. The flask was heated to 80° C. With good agitation, 30% hydrogen peroxide (3.0 g, 0.026 mol) was added over a period of 45 minutes. The flask was heated with good stirring overnight at 80° C. When 0.59 g of the solution was evaporated at 60° C. for one hour, 0.17 g of residue was obtained (solids=28.8%)

### Synthesis of N,N-diethyl-3-((1,1,2,2,3,3,4,4,4-nonafluoro-N-(2-hydroxyethyl)butyl)sulfonamido)propan-1-amine oxide (19)

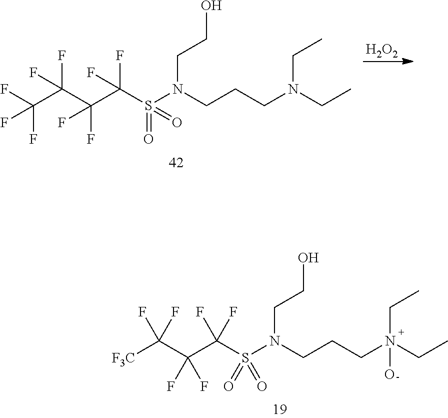

A three-necked 1 L flask equipped with an overhead stirrer, thermocouple, addition funnel, and reflux condenser was charged with N-(3-(diethylamino)propyl)-1,1,2,2,3,3,4,4,4-nonafluoro-N-(2-(2-(hydroxyethyl) butane-1-sulfonamide (42, 16 g, 35 mmol), and 32 g of isopropanol. The flask was heated to 65° C. With good agitation, 6.8 g of 30% hydrogen peroxide (6.8 g, 59 mmol) was added over a period of 45 minutes. The flask was heated with good stirring overnight at 65° C. LC/MS was consistent for the desired structure

Compounds 20-23 were synthesized from the corresponding tertiary amines according to the procedure described for the synthesis of Compound 13.

### Synthesis of N,N-diethyl-N-methyl-2-((1,1,2,2,3,3,4,4,4-nonafluoro-N-methylbutyl)sulfonamido)ethan-1-aminium iodide (24)

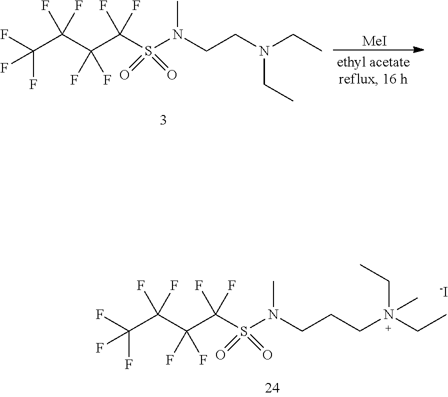

A 250 mL round bottom flask equipped with a condenser was charged with N-[2-(diethylamino)ethyl]-1,1,2,2,3,3,4,4,4-nonafluoro-N-methyl-butane-1-sulfonamide (3, 10.0 g, 24.2 mmol), 120 mL ethyl acetate, and Mel (7.0 g, 49 mmol). The reaction was heated to reflux for 16 hours and a white precipitate formed. Upon cooling to room temperature, this solid was filtered and dried under high vacuum to give a white solid (24, 12.6 g, 22.7 mmol, 94% Yield). ¹H and ¹⁹F NMR data was consistent with the desired compound.

### Synthesis of N,N-diethyl-N-(2-((1,1,2,2,3,3,4,4,4-nonafluoro-N-methylbutyl)sulfonamido)ethyl)propan-1-aminium iodide (25)

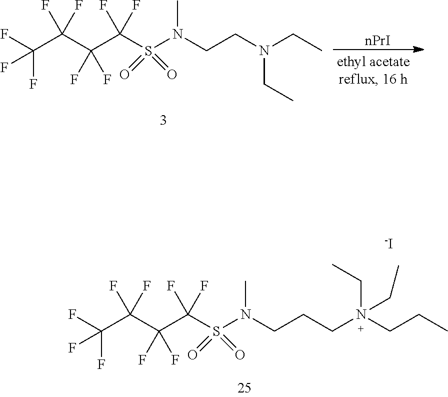

A 250 mL round bottom flask equipped with a condenser was charged with N-[2-(diethylamino)ethyl]-1,1,2,2,3,3,4,4,4-nonafluoro-N-methyl-butane-1-sulfonamide (3, 10.0 g, 24.3 mmol), 120 mL ethyl acetate, and nPrI (8.3 g, 4.8 mL, 49 mmol) was added the reaction was heated to reflux for 16 hours and a white precipitate formed. Upon cooling to room temperature, this solid was filtered and dried under high vac to give a white solid (25, 3.8 g, 6.5 mmol, 27% Yield). ¹H and ¹⁹F NMR data was consistent with the desired compound.

### Synthesis of 4-(diethyl(2-((1,1,2,2,3,3,4,4,4-nonafluoro-N-methylbutyl)sulfonamido)ethyl)ammonio)butane-1-sulfonate (26)

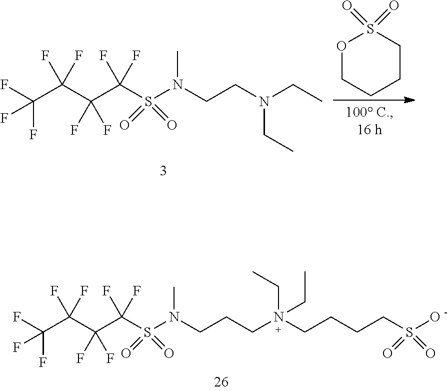

A 100 mL thick walled round bottom flask was charged with 1-methyl-4 N-[2-(diethylamino)ethyl]-1,1,2,2,3,3,4,4,4-nonafluoro-N-methyl-butane-1-sulfonamide (3, 5.0 g, 12 mmol) and oxathiane 2,2-dioxide (6.6 g, 48 mmol), sealed with a teflon plug and heated for 16 hours at 100° C., the reaction was cooled, the solid was filtered and then washed with toluene to give a white solid (26, 4.9 g, 8.9 mmol, 74% yield). ¹H and ¹⁹F NMR (in (CD₃)CO₂D) data was consistent with the desired compound.

### Synthesis of 1,1-dimethyl-4-((perfluorobutyl)sulfonyl)piperazin-1-ium iodide (27)

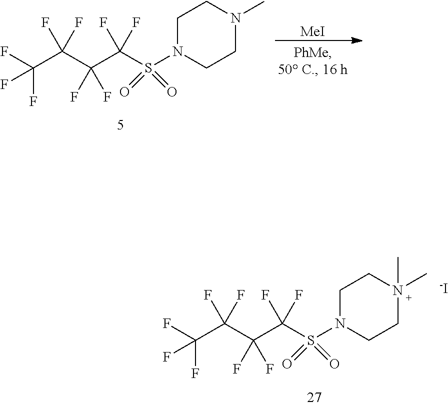

A 25 mL thick walled round bottom flask was charged with 1-methyl-4-((perfluorobutyl)sulfonyl)piperazine (5, 5.0 g, 13 mmol), toluene (10 mL), and Mel (2.0 g, 0.88 mL, 14 mmol), sealed with a teflon plug and heated for 16 hours at 50° C., the reaction was cooled, the solid was filtered and then washed with toluene to give a white solid (27, 6.60 g, 12.6 mmol, 97% yield). ¹H and ¹⁹F NMR data (in CO(CD₃)₂) was consistent with the desired compound.

### Synthesis of 1-methyl-4-((perfluorobutyl)sulfonyl)-1-propylpiperazin-1-ium bromide (28)

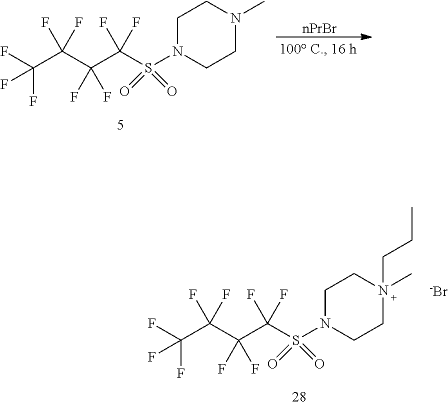

A 25 mL thick walled round bottom flask was charged with 1-methyl-4-((perfluorobutyl)sulfonyl)piperazine (5, 5.0 g, 13 mmol) and nPrBr (3.4 g, 2.6 mL, 28 mmol), sealed with a teflon plug and heated for 16 hours at 100° C., the reaction was cooled, the solid was filtered and then washed with toluene to give a white solid (28, 1.05 g, 2.47 mmol, 19% yield). ¹H and ¹⁹F NMR data (in CO(CD₃)₂) was consistent with the desired compound.

### Synthesis of 1-methyl-4-((perfluorobutyl)sulfonyl)-1-propylpiperazin-1-ium iodide (29)

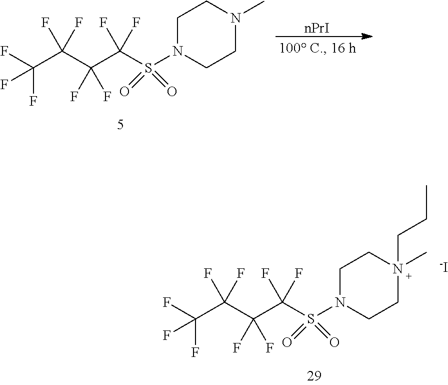

A 25 mL thick walled round bottom flask was charged with 1-methyl-4-((perfluorobutyl)sulfonyl)piperazine (5, 5.0 g, 13 mmol) and nPrI (4.8 g, 2.8 mL, 28 mmol), sealed with a teflon plug and heated for 16 hours at 100° C., the reaction was cooled, the solid was filtered and then washed with toluene to give a white solid (29, 2.3 g, 5.3 mmol, 41% yield). ¹H and ¹⁹F NMR data (in CO(CD₃)₂) was consistent with the desired compound.

### Synthesis of 1-butyl-1-methyl-4-((perfluorobutyl)sulfonyl)piperazin-1-ium iodide (30)

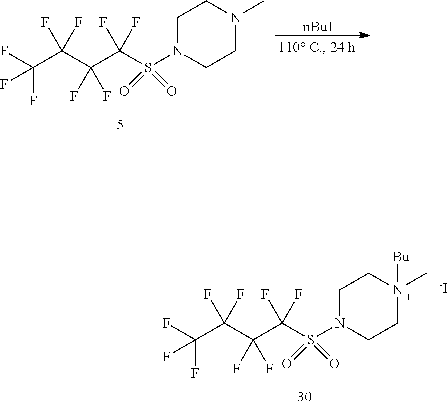

A 25 mL thick walled round bottom flask was charged with 1-methyl-4-((perfluorobutyl)sulfonyl)piperazine (5, 5.0 g, 13 mmol) and nBuI (4.8 g, 3.0 mL, 26 mmol), sealed with a teflon plug and heated for 24 h at 110° C., the reaction was cooled, the solid was filtered and then washed with toluene and ethyl acetate to give a white solid (30, 3.9 g, 6.89 mmol, 53% yield). ¹H and ¹⁹F NMR data (in CO(CD₃)₂) was consistent with the desired compound.

### Synthesis of 1-ethyl-4-((perfluorobutyl)sulfonyl)-1-propylpiperazin-1-ium iodide (31)

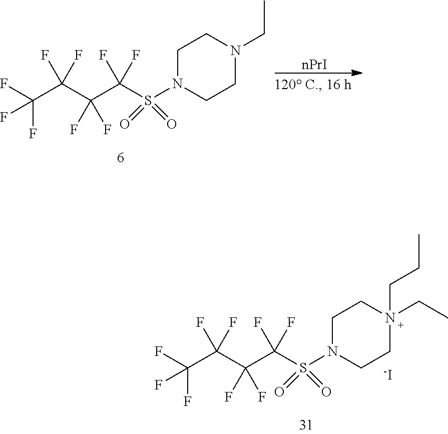

A 25 mL thick walled round bottom flask was charged with 1-ethyl-4-((perfluorobutyl)sulfonyl)piperazine (6, 5.0 g, 13 mmol), and nPrI (4.2 g, 2.4 mL, 25 mmol), sealed with a teflon plug and heated for 24 h at 120° C., the reaction was cooled, the solid was filtered and then washed with toluene and dried under high vacuum to give a white solid (31, 2.5 g, 4.4 mmol, 35% yield). ¹H and ¹⁹F NMR data (in CO(CD₃)₂) was consistent with the desired compound.

### Synthesis of 4-((perfluorobutyl)sulfonyl)-1,1-dipropylpiperazin-1-ium iodide (32)

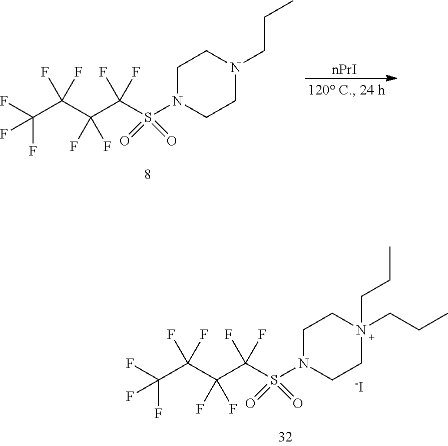

A 25 mL thick walled round bottom flask was charged with 1-((perfluorobutyl)sulfonyl)-4-propylpiperazine (8, 5.0 g, 12 mmol) and nPrI (4.2 g, 2.4 mL, 25 mmol), sealed with a teflon plug and heated for 24 h at 120° C., the reaction was cooled diluted with toluene, the solid was filtered and then washed with toluene. This solid was then suspended in DI H₂O and stirred for 16 hours, filtered, and dried under high vacuum to give a light tan solid (32, 2.1 g, 3.6 mmol, 30% yield). ¹H and ¹⁹F NMR data (in CO(CD₃)₂) was consistent with the desired compound.

### Synthesis of 1-methyl-1-octanoyl-4-((perfluorobutyl)sulfonyl)piperazin-1-ium iodide (33)

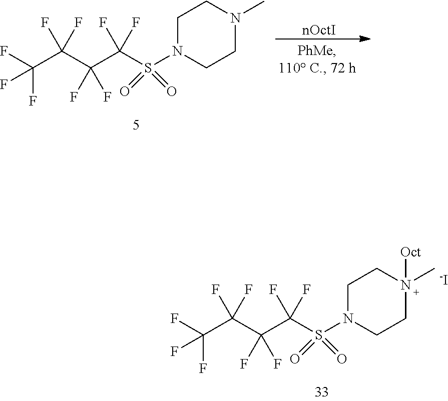

A 25 mL thick walled round bottom flask was charged with 1-methyl-4-((perfluorobutyl)sulfonyl)piperazine (5, 5.0 g, 13 mmol), 10 mL toluene, and nOctI (6.2 g, 4.7 mL, 26 mmol), sealed with a teflon plug and heated for 72 h at 110° C., the reaction was cooled, the solid was filtered and then washed with toluene and ethyl acetate to give a white solid (33, 1.67 g, 2.73 mmol, 21% yield). ¹H and ¹⁹F NMR data (in CO(CD₃)₂) was consistent with the desired compound.

### Synthesis of N,N,N-trimethyl-3-((perfluorobutyl)sulfonamido)propan-1-aminium iodide (34)

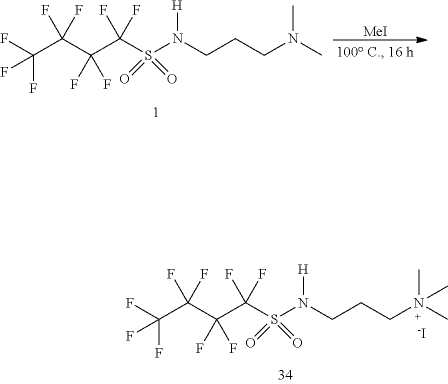

A 25 mL thick walled round bottom flask was charged with N-(3-(dimethylamino)propyl)-1,1,2,2,3,3,4,4,4-nonafluorobutane-1-sulfonamide (1, 2.5 g, 6.5 mmol) and Mel (3.0 g, 1.3 mL, 21 mmol), sealed with a teflon plug and heated for 16 hours at 100° C., the reaction was cooled, the solid was filtered and then washed with toluene to give a white solid (34, 3.3 g, 6.3 mmol, 96% yield). ¹H and ¹⁹F NMR data (in CO(CD₃)₂) was consistent with the desired compound.

### Synthesis of N,N-diethyl-N-methyl-3-((1,1,2,2,3,3,4,4,4-nonafluoro-N-methylbutyl)sulfonamido)propan-1-aminium iodide (35)

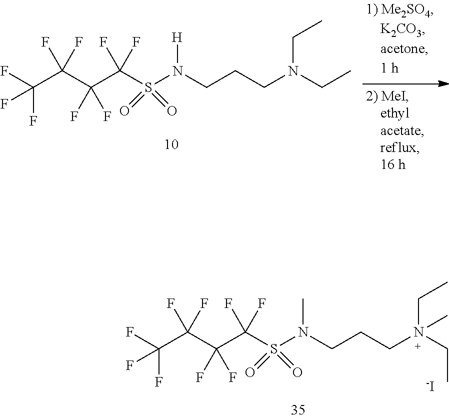

A 250 mL round bottom flask was charged with N-[3-(diethylamino)propyl]-1,1,2,2,3,3,4,4,4-nonafluoro-butane-1-sulfonamide (10, 10 g, 24.3 mmol), K₂CO₃ (7.0 g, 51 mmol), 120 mL acetone, and dimethyl sulfate (3.6 g, 2.7 mL, 29 mmol), this was stirred for 1 h at room temperature after which 100 mL DI H₂O was added and the reaction was stirred for ˜20 min, then 120 mL ethyl acetate was added and the layers were separated. The aqueous layer was again washed with 100 mL ethyl acetate (a small amount of brine can be useful to break up the emulsion that forms) the combined organic phases were dried over Na₂SO₄, filtered, and concentrated in vacuo to give a light-yellow oil that was immediately dissolved in 120 mL of ethyl acetate in a 250 mL flask equipped with a condenser; Mel (8.0 g, 3.5 mL, 56 mmol) was then added to the resulting solution, and the reaction was refluxed for 16 hours. The reaction was concentrated to give a tan solid that was dried and then stirred in 50 mL ethyl acetate for ˜1 h, filtered to give a fine white powder (35. 9.7 g, 17 mmol, 70% Yield). ¹H and ¹⁹F NMR data was consistent with the desired compound.

### Synthesis of N,N,N,2,2-pentamethyl-3-((1,1,2,2,3,3,4,4,4-nonafluoro-N-methylbutyl)sulfonamido)propan-1-aminium iodide (36)

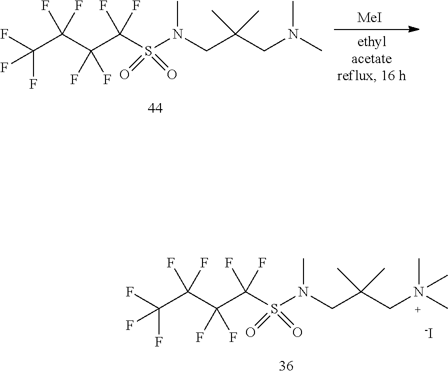

A 250 mL round bottom flask equipped with a condenser was charged with N-[3-(dimethylamino)-2,2-dimethyl-propyl]-1,1,2,2,3,3,4,4,4-nonafluoro-N-methyl-butane-1-sulfonamide (44, 8.4 g, 20 mmol), 100 mL ethyl acetate, and Mel (5.5 g, 2.4 mL, 39 mmol) was added the reaction was heated to reflux for 16 hours. Upon cooling to room temperature a solid precipitated, this was filtered and dried under high vacuum to give a white solid (36, 8.7 g, 15 mmol, 78% Yield). ¹H and ¹⁹F NMR data was consistent with the desired compound.

### Surface Tensions of C-4 Amine Oxides

In the following tables, examples are designated as EX- and comparative examples are designated as CE-.

Table 3 below shows the surface tensions of the amine oxides: N-alkyl-N-(dialkylamino)alkyl)-perfluorobutanesulfonamido amine oxides:

C₄F₉SO₂N(R)A-N(R′)(R″)(O)

where A is alkylene group (CH₂)ₙ, or a cyclic moiety attaching the two nitrogens, n is an integer from 2-6, R=alkyl groups containing 1-6 carbons or alkoxy groups containing 1-8 carbons, and R′ and R″ are alkyl groups containing 1-6 carbons at 2000 ppm in three solution, DI water, 2.5% tetramethylammonium hydroxide, and 5% HCl. Comparative perfluorobutanesulfonamido cationic and zwitterion surfactant surface tensions also at 2000 ppm are listed in Table 4. No perfluorooctyl or perfluorohexyl surfactant were made because those longer perfluoro chains are considered too bioaccumulative to be viable commercial products. Comparing the surface tensions in Tables 3 and 4, one can see that the C-4 amine oxides are a superior group of cationic/zwitterionic surfactants among the other ones with a C-4 perfluorinated tail.

TABLE 3
Surface Tensions of Amine Oxides at 2000 ppm (dynes/cm)

| | Compound | | | 2.5% | 2.5% |
|---|---|---|---|---|---|
| Example | # | Structure | Water | TMAH | HCl |
| EX-1 | 11 | 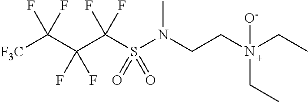 | 20.7 | 22 | 21 |
| EX-2 | 12 | 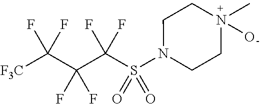 | 19.2 | 24.5 | 22 |
| EX-3 | 13 | 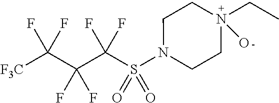 | 24 | 25 | 21 |
| EX-4 | 14 | 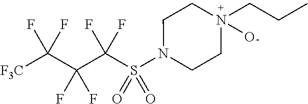 | 20 | 25 | 22 |
| EX-5 | 15 | 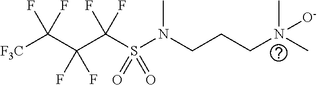 | 17.9 | 21.8 | 23* |
| EX-6 | 16 | 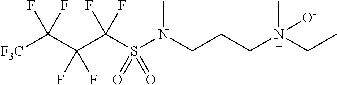 | 21.7 | 22.9 | 24.8 |
| EX-7 | 17 | 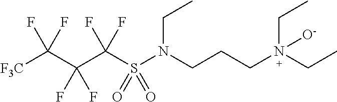 | 21.9 | 25 | 22.6 |
| EX-8 | 18 | 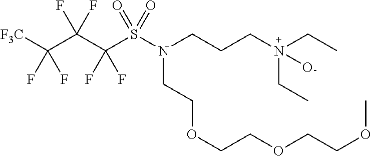 | 22.5 | 23.7 | 25.1 |
| EX-9 | 19 | 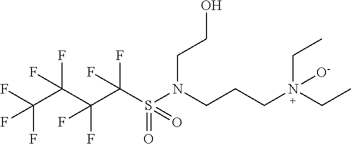 | 20 | 26 | 25 |

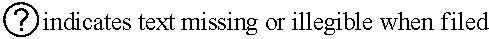

*Not completely soluble

TABLE 4
Surface Tensions of Perfluorobutanesulfonamido Comparative Examples 2000 ppm (dynes/cm)

| | Compound | | | 2.5% | 2.5% |
|---|---|---|---|---|---|
| Example | # | Structure | Water | TMAH | HCl |
| CE-1 | 20 | 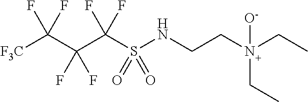 | insoluble | — | — |
| CE-2 | 21 | 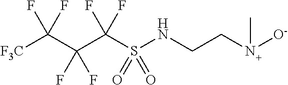 [SMILES: CN([O-])CCNS(=O)(=O)C(F)(F)C(F)(F)C(F)(F)C(F)(F)F] | 18.5 | 28 | 22 |
| CE-3 | 22 | 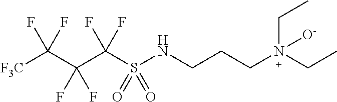 | 22.1 | 35.1 | 22 |
| CE-4 | 23 | 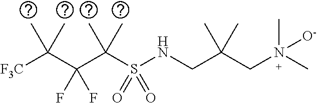 | insoluble | — | — |
| CE-5 | 24 | 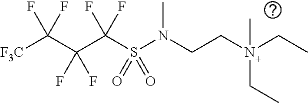 | 44.5 | — | — |
| CE-6 | 25 | 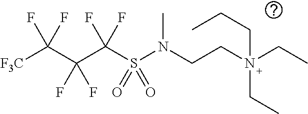 | 39 | 38 | — |
| CE-7 | 26 | 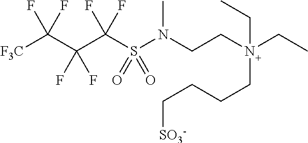 | 42.7 | 34.4 | — |
| CE-8 | 27 | 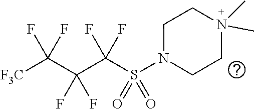 | 43.3 | 26.5 | — |
| CE-9 | 28 | 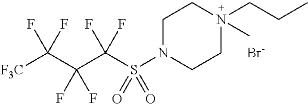 | 46.1 | 32.7 | — |
| CE-10 | 29 | 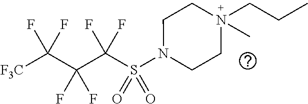 | 30.4 | 35.5* | — |
| CE-11 | 30 | 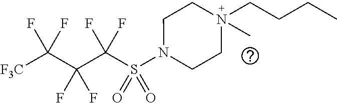 | 35.8 | 35.8 | — |
| CE-12 | 31 | 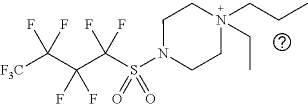 | 38 | 42 | 37 |
| CE-13 | 32 | 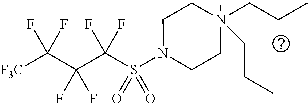 | 32 | 25 | 40 |
| CE-14 | 33 | 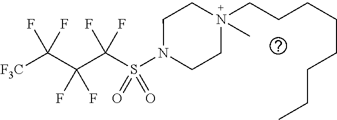 | 32.4* | 26.8* | — |
| CE-15 | 34 | 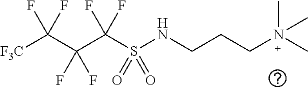 | 48.8 | 35.5 | — |
| CE-16 | 35 | 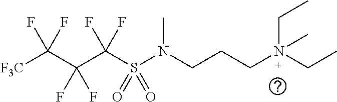 | 41.2 | 30.3 | — |
| CE-17 | 36 | 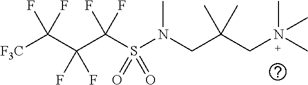 | 38.7 | 34.9 | — |

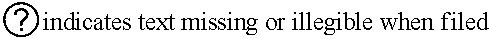

*Not completely soluble

Table 5 shows amine oxide surfactants and comparative examples at 250 parts per million (ppm) in water, 2.5% tetramethyl ammonium hydroxide, 2.5% HCl, and 50% sulfuric acid.

TABLE 5
Surface Tensions at 250 ppm

| | | | | 2.5% | 2.5% | 50% |
|---|---|---|---|---|---|---|
| Example | # | Structure | Water | TMAH | HCl | sufuric |
| EX-7 | 17 | 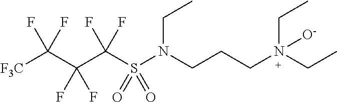 | 20.9 | 32.7 | 25.1 | 24.9 |
| CE-3 | 22 | 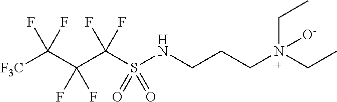 | 25.6 | — | — | — |
| EX-5 | 15 | 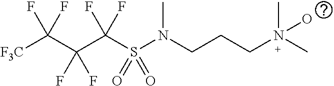 | 27.9 | 36.4 | 31.5 | 27.5 |
| EX-1 | 11 | 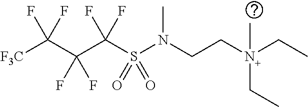 | 33.5 | — | — | — |
| EX-8 | 18 | 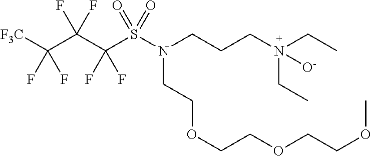 | 34.4 | — | — | — |
| EX-3 | 13 | 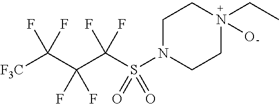 | 38.8 | — | — | — |
| CE-2 | 21 | 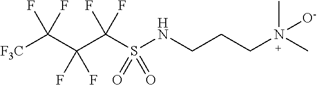 | — | — | — | 37 |

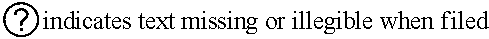

1. An aqueous surfactant composition comprising a surfactant of the formula: 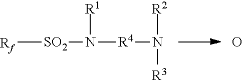
wherein R_{f} is a perfluoroalkyl group, each of R¹, R² and R³ are C₁-C₈ alkyl, alkoxy, or aryl; R⁴ is an arylene or an alkylene having 1 to 20 carbon atoms, preferably 2-8, said alkyl and alkylene may be cyclic or acyclic, may optionally contain catenated (in-chain) nitrogen heteroatoms; and ii) an aqueous solvent; said composition having less than 1000 ppb of ionic contaminants.

2. The composition of claim 1, wherein R_{f} is from 3 to 5 carbon atoms.

3. The composition of claim 1, wherein the surfactant is present at a concentration of at least 0.001 wt. % of the composition.

4. The composition of claim 1, wherein the surfactant is present at a concentration of up to 1 weight percent of the composition.

5. The composition of claim 1 having less than 500 ppb of ionic contaminants.

6. The composition of claim 1, wherein the optional solvent is a water-soluble organic solvent.

7. The composition of claim 1 comprising less than 1 wt % organic solvent.

8. The composition of claim 1, wherein said composition further comprises iii) one or more additives selected from the group consisting of abrasive particles, other acids, oxidizing agents, etchants, corrosion inhibitors, chelating agents, electrolytes, surfactants, brighteners, and levelers.

9. A composition comprising: a) at least 0.001 weight percent of the surfactant of claim 1; b) optional aqueous solvent; and c) oxidizing agent.

10. The composition of claim 9, wherein said oxidizing agent is selected from the group consisting of nitric acid, HNO₃, H₂O₂, Fe(NO₃)₃, O₃, and mixtures thereof

11. The aqueous surfactant composition of claim 1 comprising 0.001-0.5 wt. % range of the surfactant, 1-10, preferably 3-5 wt. % of NH₄OH, 1-10, preferably 3-5 wt. % of H₂O₂ and deionized water.

12. (canceled)

13. An aqueous surfactant composition of claim 1 comprising 0.001-0.5 wt. % of the surfactant of Formula I, 0.25-10, preferably 0.5-5 wt. % of HF and deionized water.

14. (canceled)

15. An aqueous surfactant composition of claim 1 (SC-2 cleaning composition) comprising 0.001-0.5 wt. % of the surfactant of Formula I, 1-10, preferably 4-6 wt. % range of HCl, wt. %, 1-10, preferably 3-5 wt. % of H₂O₂ and deionized water.

16.-17. (canceled)

18. A CMP slurry composition comprising the composition of claim 1 comprising 0.001 to 0.5 wt. % of the surfactant, 1-10 wt. % of organic acid, 1-10 wt. % of H₂O₂, an aqueous solvent, and an abrasive particle.

19. The CMP slurry of claim 18 wherein the organic acid is selected from citric acid, oxalic acid, succinic acid and an alkyl sulfonic acid.

20. The composition of claim 1 comprising 10-15 wt. % H₂O₂; 25-50 wt. % H₂SO₄; and at least 0.001 wt. % of the surfactant.

21. A process of etching comprising contacting a substrate with the composition of claim 20.

22. A composition of claim 1, wherein the amine oxide is prepared by treatment of the precursor amine with peroxide.

23. The composition of claim 22 wherein the precursor amine is of the formula 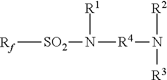
wherein R_{f} is a perfluoroalkyl group, each of R¹, R² and R³ are C₁-C₈ alkyl, alkoxy or aryl; R⁴ is an arylene or an alkylene having 1 to 20 carbon atoms, preferably 2-8, said alkyl and alkylene may be cyclic or acyclic, may optionally contain catenated nitrogen heteroatoms.

24. The composition of claim 23 wherein the precursor amine is distilled prior to oxidation.
